(19) United States
(12) Patent Application Publication
(10) Pub. No.: US 2006/0160223 A1
(43) Pub. Date: Jul. 20, 2006

(54) Plasmid vectors for transformation of filamentous fungi

(76) Inventors: Thierry Lacour, Stutensee (DE); Jan Rether, Kaiserslautern (DE); Annette Freund, Limburgerhof (DE); Ralf-Michael Schmidt, Kirrweiler (DE); Wilhelm Schafer, Hamburg (DE); Frank Maier, Jork (DE); Sascha Malz, Hamburg (DE)

Correspondence Address: CONNOLLY BOVE LODGE & HUTZ, LLP, P O BOX 2207, WILMINGTON, DE, 19899, US

(21) Appl. No.: 10/519,210

(22) PCT Filed: Jul. 2, 2003
(86) PCT No.: PCT/EP03/07028

§ 371 Date: Dec. 23, 2004

(30) Foreign Application Priority Data
Jul. 5, 2002 (EP) 02015067.8
Apr. 11, 2003 (EP) 03008444.6

(51) Int. Cl. C12N 1/16; C12N 15/74

(52) U.S. Cl. 435/484; 435/254.3

(57) ABSTRACT

The invention relates to novel plasmid vectors for transformation of filamentous fungi and to a method of modifiying the genome of filamentous fungi based on these vectors. The invention furthermore relates to the modification of a specific gene via the process of homologous recombination, to recombinant expression of foreign genes in filamentous fungi and to new selection markers for detecting succesfull transfer of the target gene in filamentous fungi.

The invention relates to novel plasmid vectors for transformation of filamentous fungi and to a method of modifiying the genome of filamentous fungi based on these vectors. The invention furthermore relates to the modification of a specific gene via the process of homologous recombination, to recombinant expression of foreign genes in filamentous fungi and to new selection markers for detecting the successful transfer of the target gene in filamentous fungi.

One method currently used for transformation of filamentous fungi is random mutagenesis based on transposons insertion, a method also known for plant transformation (WO 01/38509). This method allows the genomes of several species such as Magnaporthe grisea to be studied (for examples WO 00/55346; WO 00/56902). However, this strategy requires a big effort in terms of bioinformatic tools and molecular biology to localise precisely the insertion in the genome.

Alternatively, known transformation methods are based on targeted integration. Targeted transformation of fungi can be carried out either by offering a knock-out cassette with a marker-gene flanked by two homologous sequences (Aronson et al, 1994, Mol. Gen. Genet. 242: 490-494; Royer et al, 1999, Fungal Genetics and Biology 28: 68-78; Schaefer, 2001, Current Opinion in Plant Biology 4: 143-150) or by quoting a plasmid with the marker gene in the neighburship of a homologous sequence (Shortle et al., 1982, Science 217: 371-373; Bird and Bradshaw, 1997, Mol Gen Genet. 255: 219-225; Feng et al., 2001, Infection and Immunity 69 (3): 1781-1794; Schaefer, 2001, Current Opinion in Plant Biology 4: 143-150). Both procedures are in principle attractive methods to study the gene function, but they have the disadvantage of a high frequency of integration at ectopic sites by illegitimate recombination. The gene targeting efficiency (gene targeting/gene targeting+illegitimate recombination) is 95% for S. cerevisiae, 10-90% for S. pombe, 5-75% for Aspergillus nidulans and 1-30% for Neuropsora crassa using a size of homology of 2-9 Kb (Schaefer, 2001, Current Opinion in Plant Biology 4: 143-150). Especially for filamentous fungi this side effect is quite high, if conventional plasmid vectors are used.

In addition, the efficiency of the gene targeting increases if the length of homologous recombination region is increased (Schaefer, 2001, Current Opinion in Plant Biology 4: 143-150). Thus, plasmid vectors currently used comprise gene fragments of the gene to be knocked out of a size of at least 2000 bp as indicated above. The overall size of these plasmid vectors is at least 8000 bp (P. J. Punt et. al., 1992, Methods in Enzymology, vol. 216, pp 447-457; Schaefer, 2001, Current Opinion in Plant Biology 4: 143-150). Since transformation efficiency decreases with the increase of the plasmid vector size, transformation efficiency is unsatisfactory giving rise to long times until positive clones can be identified. This is an obstacle especially to large scale genomic analysis projects or recombinant expression. Furthermore, currently used plasmid vectors contain many unique restriction sites, causing difficulties in construction of the knock-out (KO-) plasmids and the transformation process. The efficiency of homologous recombination is improved when the KO-plasmid is digested with a restriction enzyme presenting a unique site in the middle of a DNA fragment homologous to the targeted gene. The presence of high amounts of restriction sites especially unique ones in the plasmid backbone decreases the chance of finding a natural restriction site in the appropriate location of the targeted DNA fragment. This problem is usually overcome by modification of the targeted DNA fragment requiring several cloning steps and additional manipulation in terms of molecular biology, which is a disadvantageous and time consuming methology.

Integration of recombinant gen by homolgous recombination in fungi is also a tool to identify gene functions for essential genes: the biochemical characterization of an essential gene cannot be studied by classical knock-out strategy since the mutants carrying a disruption of such a gene are not viable. One way to overexpress such a gene overcomes the problem when a typical phenotype can be assigned to the mutant that overexpresses the gene. Another approach can be to regulate the gene expression by an inducible promoter sequence so that the gene could be expressed or repressed when needed and consequently permits viable mutants to be isolated. As mentioned above, these approaches require at least several thousand bp of the nucleic acid sequence to be studied that need to be integrated in the genome of the fungi together with a plasmid vector comprising the different parts of the nucleic acid sequence. In addition, if the recombinant DNA is integrated at an ectopic site, the identification of the mutant strains becomes more complicated and the position of the integration in the genome may influence the level of expression of the recombinant protein. Taking the aforesaid into consideration, currently existing plasmid vectors currently used for transformation of filamentous fungi exhibit a lot of disadvantages and are neither suitable for large scale analysis e.g. in functional genomic studies nor convenient for recombinant expression in a filamentous fungi. Additionally, there is a constant need for new selection markers facilitating the selection process.

Thus, object of the present invention was to develop tools for targeted transformation of filamentous fungi that overcome the disadvantages of the state of the art like plasmid vectors suitable for functional genomic studies and recombinant expression and new selection markers.

We have found that the object of the invention is achieved by construction of a plasmid vector for targeted transformation of filamentous fungi comprising
a) an origin of replication for a host organism not originating from the filamentous fungi to be transformed;
b) a selection marker for a host organism not originating from the filamentous fungi to be transformed;
c) a promotor facilitating recombinant expression in filamentous fungi that is functionally linked to the coding region of the hygromycin resistance gene which is functionally linked to a terminator which facilitates transcription termination in filamentous fungi;
   wherein the overall size of the elements a), b) and c) does not exceed 4500 bp; and
d) a nucleic acid sequence, which is homologous to nucleic acid sequences of the filamentous fungi to be transformed and makes homologous recombination in the filamentous fungi to be transformed possible.

The term overall size of the elements a), b) and c) designates the combination of the essential elements of the expression vector without the nucleic acid sequence d).

The overall size of the elements a), b) and c) does not exceed 4500 bp, preferaby 4100 bp, more preferably 3700 bp.

In addition to the nucleic acid elements a), b), c) and d), the plasmid vector optionally comprises a cloning site containing rare restriction sites or a TA-cloning site by which further nu-cleic acid sequences can be cloned easily into the plasmid vector. A TA-cloning site comprises thymidine residues linked onto the 3′-ends of linearized plasmid DNA, which would allow some annealing to occur between the vector and the A-tailed PCR product to be ligated. This process is called TA cloning. Preferably, the vector is modified in such a way that there are only few unique restriction sites left enabling the digestion by commercially available restriction enzymes of the homologous sequence of the targeted gene prior to the transformation.

Filamentous fungi that can be transformed with the vectors of the present invention are non-phytopathogenic filamentous fungi e.g. Neurospora species like Neurospora crassa and phytopathogenic filamentous fungi the phytopathogenic filamentous fungi being preferred. Examples of other non-pythopathogenic filamentous fungi are Aspergillus species such as Aspergillus parasiticus, Aspergillus nidulans, Aspergillus niger and Wagiella such as Wangiella dermatidis. Preferred phytopathogenic filamentous fungi are selected from the group consisting of the genera Alternaria, Podosphaera, Sclerotinia, Physalospora, Botrytis, Corynespora; Colletotrichum; Diplocarpon; Elsinoe; Diaporthe; Sphaerotheca; Cinula, Cercospora; Erysiphe; Sphaerotheca; Leveillula; Mycosphaerella; Phyllactinia; Gloesporium; Gymnosporangium, Leptotthrydium, Podosphaera; Gloedes; Cladosporium; Phomopsis; Phytopora; Phytophthora; Erysiphe; Fusarium; Verticillium; Glomerella; Drechslera; Bipolaris; Personospora; Phaeoisariopsis; Spaceloma; Pseudocercosporella; Pseudoperonospora; Puccinia; Typhula; Pyricularia; Rhizoctonia; Stachosporium; Uncinula; Ustilago; Gaeumannomyces and Fusarium, more preferred from the group consisting of the genera and species Alternaria, Podosphaera, Sclerotinia, Physalospora such as Physalospora canker, Botrytis species such as Botrytis cinerea, Corynespora such as Corynespora melonis; Colletotrichum; Diplocarpon such as Diplocarpon rosae; Elsinoe such as Elsinoe fawcetti, Diaporthe such as Diaporthe citri; Sphaerotheca; Cinula such as Cinula neccata, Cercospora; Erysiphe such as Erysiphe cichoracearum and Erysiphe graminis; Sphaerotheca such as Sphaerotheca fuliginea; Leveillula such as Leveillula taurica; Mycosphaerella; Phyllactinia such as Phyllactinia kakicola; Gloesporium such as Gloesporium kaki; Gymnosporangium such as Gymnosporangium yamadae, Leptotthrydium such as Leptotthrydium pomi, Podosphaera such as Podosphaera leucotricha; Gloedes such as Gloedes pomigena; Cladosporium such as Cladosporium carpophilum; Phomopsis; Phytopora; Phytophthora such as Phytophthora infestans; Verticillium; Glomerella such as Glomerella cingulata; Drechslera; Bipolaris; Personospora; Phaeoisariopsis such as Phaeoisariopsis vitis; Spaceloma such as Spaceloma ampelina; Pseudocercosporella such as Pseudocercosporella herpotrichoides; Pseudoperonospora; Puccinia; Typhula; Pyricularia such as Pyricularia oryzae; Rhizoctonia; Stachosporium such as Stachosporium odorum; Uncinula such as Uncinula necator; Ustilago; Gaeumannoyces species such as Gaeumannomyces graminis and Fusarium such as Fusarium dimerium, Fusarium merismoides, Fusarium lateritium, Fusarium decemcellulare, Fusarium poae, Fusarium tricinctum, Fusarium sporotrichioides, Fusarium chlamydosporum, Fusarium moniliforme, Fusarium proliferatum, Fusarium anthophilum, Fusarium subglutinans, Fusarium nygamai, Fusarium oxysporum, Fusarium solani, Fusarium culmorum, Fusarium sambucinum, Fusarium crookwellense, Fusarium avenaceum ssp. avenaceum, Fusarium avenaceum ssp. aywerte, Fusarium avenaceum ssp. nurragi, Fusarium hetrosporum, Fusarium acuminatum ssp. acuminatum, Fusarium acuminatum ssp. areniacum, Fusarium longipes, Fusarium compactum, Fusarium equiseti, Fusarium scripi, Fusarium polyphialidicum, Fusarium semitectum and Fusarium beomiforme and especially preferred from the genera Fusarium such as Fusarium graminearum, most preferred from the group consisting of the genera and species Fusarium, Fusarium dimerium, Fusarium merismoides, Fusarium lateritium, Fusarium decemcellulare, Fusarium poae, Fusarium tricinctum, Fusarium sporotrichioides, Fusarium chlamydosporum, Fusarium moniliforme, Fusarium proliferatum, Fusarium anthophilum, Fusarium subglutinans, Fusarium nygamai, Fusarium oxysporum, Fusarium solani, Fusarium culmorum, Fusarium sambucinum, Fusarium crookwellense, Fusarium avenaceum ssp. avenaceum, Fusarium avenaceum ssp. aywerte, Fusarium avenaceum ssp. nurragi, Fusarium hetrosporum, Fusarium acuminatum ssp. acuminatum, Fusarium acuminatum ssp. armeniacum, Fusarium longipes, Fusarium compactum, Fusarium equiseti, Fusarium scripi, Fusarium polyphialidicum, Fusarium semitectum and Fusarium beomiforme wherein Fusarium graminearum is most preferred.

The host organism in which the origin of replication a) is functionally active essentially serves to construct and propagate the plasmid vector of the invention. The host organism must be genetically different from the filamentous fungi to be transformed, since replication of the plasmid vector should not take place in the filamentous fungi to be transformed but is desired in the host organism, due to the use of the origin of replication a). Host organisms which may be used are all common microorganisms which can easily be manipulated by genetic engineering. Preferred host organisms are Gram-negative bacteria such as the genera Escherichia and Salmonella e.g. Escherichia coli and Salmonella thyplimurium or Gram-positive bacteria such as the genera Bacillus and Streptomyces, e.g. Bacillus subtilis and Streptomyces nidulans. Particularly preferred are gram-negative bacteria such as Escherichia, e.g. Escherichia coli.

Preferred origins of replication (ori) are the col E1 ori, the f1 ori.

The term “selection marker for a host organism” set forth in b) means a gene or the expression product of the gene. Preferred meanings are genes whose expression causes resistance of the host organism to antibiotics, by preference resistance to kanamycin, chloramphenicol, tetracycline, zeocin or ampicillin, and particularly preferred ampicillin and kanamycin.

In a preferred embodiment, the element a) of the plasmid vector according to the invention comprises a col E1 origin of replication and the ampicillin resistance gene as selection marker for the host organism.

The element c) is hereinbelow termed “hygromycin cassette”. The coding region of the hygromycin resistance gene (hereinbelow termed “hygromycin gene”) is known by the skilled artisan (Gritz L. and Davies J., 1983, Gene 25, 179-188, Kaster, K. R., Burgett S. G. and Ingolia T. D., 1984, Curr. Genet. 8,353-358) and has a length of 1026 bp.

Examples of suitable promoters to which the coding region of the hygromycin gene is functionally linked, are the GPD-1-, PX6-, TEF-, CUP1-, PGK-, GAP1-, TPI, PHO5-, AOX1, GAL10/CYC-1, CYC1, OliC-, ADH-, TDH-, Kex2-, MFα-, or the NMT-promotor (Degryse et al., Yeast 1995 Jun. 15; 11(7):629-40; Romanos et al. Yeast 1992 June;8(6):423-88; Benito et al. Eur. J. Plant Pathol. 104, 207-220 (1998); Cregg et al. Biotechnology (N Y) 1993 August;11(8):905-10; Luo X., Gene 1995 September. 22; 163(1):127-31; Nacken et al., Gene 1996 October. 10; 175(1-2):253-60; Turgeon et al., Mol Cell Biol 1987 September;7(9):3297-305), preferably the CYC1-, ADH-, TDH-, Kex2-, GPD-1-, PX6, TEF-, CUP1-, PGK-, GAP1-, TPI, PHO5- or AOX1-promotor, more preferably the GPD-1-, PX6, TEF- or the CUP1-promotor, most preferably the GPD1 or the TEF-promotor.

Examples of suitable terminators that are functionally linked to the coding region of the hygromycin gene are the AOX1-, nos-, PGK-, TrpC- or the CYC1-terminator (Degryse et al., Yeast 1995 Jun. 15; 11(7):629-40; Brunelli et al. Yeast 1993 December9(12): 1309-18; Frisch et al., Plant Mol. Biol. 27 (2), 405-409 (1995); Scorer et al., Biotechnology (N.Y.) 12 (2), 181-184 (1994), Genbank acc. number Z46232; Punt et al., (1987) Gene 56 (1), 117-124)), preferably the CYC1- or nos-terminator, more preferably the nos-terminator.

In a preferred embodiment, the hygromycin cassette comprises a GPD-1 promotor functionally linked to the coding region of the hygromycin region which is functionally linked to the nos-terminator.

A functional linkage is understood to mean the sequential arrangement of promoter and coding sequence, of coding sequence and terminator or of promoter, coding sequence and terminator in such a manner that each of the regulatory elements can, upon expression of the coding sequence, fulfil its function for the recombinant expression of the nucleic acid sequence. Direct linkage in the chemical sense is not necessarily required for this purpose. Preferred arrangements are those in which the hygromycin gene to be expressed recombinantly is positioned downstream of the sequence which acts as promoter, so that the two sequences are linked covalently to each other. The distance between the promoter sequence and the nucleic acid sequence to be expressed recombinantly is preferably less than 100 base pairs, especially preferably less than 50 base pairs, and very especially preferably less than 10 base pairs. The distance between the terminator sequence and the nucleic acid sequence to be expressed recombinantly is preferably less than 100 base pairs, especially preferably less than 50 base pairs, and very especially preferably less than 10 base pairs. However, further sequences which, for example, exert the function of a linker with certain restriction enzyme cleavage sites, or of a signal peptide, may also be positioned between the two sequences.

These vectors are not only much more smaller than the currently used plasmid vectors, but also exhibit a high transformation efficiency. Surprisingly, a high transformation efficiency can be gained even if small DNA-fragments of at least 300 bp, preferably at least 400 bp, more preferably at least 450 bp, most preferably at least 500 bp of the nucleic acid sequence d) to be analyzed are used. The average degree of illegitimate recombination is below 30%, preferably below 25%, more preferably 20% and most preferably between 0 and 15%.

The nucleic acid sequence d) has a homology of at least 80% to the nucleic acid sequence of the filamentous fungi to be transformed, preferably at least 90%, more preferably at least 95% and most preferably at least 100%.

In a preferred embodiment, the nucleic acid sequence d) originates from a filamentous fungi and has a length of at least 300 bp, preferably 400 bp, more preferably at least 450 bp, and most preferably at least 500 bp. These lengths are suitable for functional genomic studies for which a high number of transformants is required. Also nucleic acid sequences exceeding 500 bp can be used, e.g. for the purpose of recombinant expression.

If the nucleic acid sequence d) is to be expressed recombinantly in the filamentous fungi, it can be functionally linked to a promotor e) and optionally to a terminator f).

Examples of suitable promoters e) are for example the AUG1-, GPD-1-, PX6-, TEF-, CUP1-, PGK-, GAP1-, TPI, PHO5-, AOX1, GAL10/CYC-1, CYC1, OliC-, ADH-, TDH-, Kex2-, MFα- or the NMT-promotor or combinations of the aforementioned promoters (Degryse et al., Yeast 1995 Jun. 15; 11(7):629-40; Romanos et al. Yeast 1992 June;8(6):423-88; Benito et al. Eur. J. Plant Pathol. 104, 207-220 (1998); Cregg et al. Biotechnology (N Y) 1993 August;11(8):905-10; Luo X., Gene 1995 Sep. 22; 163(1):127-31; Nacken et al., Gene 1996 Oct. 10; 175(1-2): 253-60; Turgeon et al., Mol Cell Biol 1987 September;7(9):3297-305).

Examples of suitable terminators f) are the NMT-, Gcy1-, TrpC-, AOX1-, nos-, the PGK- or the CYC1-terminator (Degryse et al., Yeast 1995 Jun. 15; 11(7):629-40; Brunelli et al. Yeast 1993 December9(12): 1309-18; Frisch et al., Plant Mol. Biol. 27 (2), 405-409 (1995); Scorer et al., Biotechnology (N.Y.) 12 (2), 181-184 (1994), Genbank acc. number Z46232; Zhao et al. Genbank acc number: AF049064; Punt et al., (1987) Gene 56 (1), 117-124).

The nucleic acid sequence d) can be also functionally linked to an affinity tag to purify the encoded protein and/or to a reporter gene to study biochemical properties of the nucleic acid sequence d) in vivo, respectively.

“Reporter genes” encode readily quantifiable proteins. Using these genes, an assessment of transformation efficacy or of the site or time of expression can be made via growth, fluorescence, chemoluminescence, bioluminescence or resistance assay or via photometric measurement (intrinsic color) or enzyme activity. Very especially preferred in this context are reporter proteins (Schenborn E, Groskreutz D. Mol. Biotechnol. 1999; 13(1):29-44) such as the “green fluorescence protein” (GFP) (Gerdes H H and Kaether C, FEBS Lett. 1996; 389(1):44-47; Chui W L et al., Curr. Biol. 1996, 6:325-330; Leffel S M et al., Biotechniques. 23(5):912-8, 1997), chloramphenicol acetyl transferase, a luciferase (Giacomin, Plant Sci. 1996, 116:59-72; Scikantha, J. Bact. 1996, 178:121; Millar et al., Plant Mol. Biol. Rep. 1992 10:324-414), and luciferase genes, in general β-galactosidase or β-glucuronidase (Jefferson et al., EMBO J. 1987, 6, 3901-3907), the Ura3 gene, the Ilv2 gene, the 2-desoxyglucose-6-phosphate phosphatase gene, β-lactamase gene, the neomycin phosphotransferase gene, the hygromycin phosphotransferase gene, or the BASTA (=gluphosinate) resistance gene.

The term “affinity tag” denotes a peptide or polypeptide whose coding nucleic acid sequence can be fused to the nucleic acid sequence d) either directly or using a linker, by classical cloning techniques. The affinity tag serves to isolate the recombinant target protein by means of affinity chromatography. The abovementioned linker can optionally comprise a protease cleavage site (for example for thrombin or factor Xa), whereby the affinity tag can be cleaved off from the target protein, as required. Examples of customary affinity tags are the “his-tag”, for example from Quiagen, Hilden, “strep-tag”, “myc-tag” (Invitrogen, Carlsberg), New England Biolab's tag which consists of a chitin binding domain and an intein, and what is known as the CBD-tag from Novagen.

In a particularly preferred embodiment, the plasmid vector comprises an coli E1 ori, the ampicillin resistance gene as selection marker, a GPD-1 promotor functionally linked to the coding region of the hygromycin resistance gene which is functionally linked to the nos-terminator.

Preferably, the vector also comprises a multiple cloning site comprising an appropriate restriction enzyme site. Appropriate restriction sites are well known by the skilled artisan.

In a further preferred embodiment, the plasmid vector additionally comprises a TA-cloning site to facilitate the overall cloning procedure.

Examples of particularly preferred embodiments are set forth in FIGS. 1 and 2.

All of the above mentioned embodiments of plasmid vectors are hereinbelow termed “plasmid vector (or vector) according to the invention”.

A vector according to the invention may also comprise at least one additional selection marker.

If a plasmid is used for recombinant expression in host organisms, a marker is required indicating the sucessful transfer of the plasmid vector DNA into the filamentous fungi to be transformed.

Surprisingly, we have found that the gene fragments of the polyketide synthase are a my suitable selection marker. The term “selection marker” referring to the polyketide synthase herein means a nucleic acid sequence.

More precisely, the term “selectable marker”, “selection marker” or “marker” used in connection with polyketide synthetase for transformation of filamentous fungi means a nucleic acid sequence encoding a polyketide synthetase or fragments of the afore-mentioned nucleic acid sequence. Preferred embodiments of the aforementioned marker as well as preferred embodiments of methods of use of the respective marker are described herein below.

Polyketide synthases are multifunctional enzymes that are involved in the biosynthesis of several important polyketides. Polyketides constitute a large and highly diverse group of secondary metabolites, synthesized by bacteria, fungi and plants and algae. They include antibiotics, compounds with mycotoxic activity, and compounds within pigment biosynthetic pathways. Furthermore a polyketide synthase is described to be required for fungal virulence of Cochliobolus heterostrophous toward maize (Yang et al., 1996 PMID:8953776). Polyketide Synthetases are furthermore known from Wangiella dermatidis (pubMedID:11179356), from Aspergillus nidulans (Swiss-prot ID: Q03149) and from Aspergillus parasiticus (Swiss-Prot ID:Q12053).

The use of polyketide synthase as selectable marker for recombinant expression in filamentous fungi has not yet been described.

The present invention also encompasses a selection marker comprising a nucleic acid sequence encoding a polyketide synthetase fragment, wherein said nucleic acid sequence comprises
i. a nucleic acid sequence shown in SEQ ID NO:1 or SEQ ID NO:2; or
ii. parts of the nucleic acid sequence as defined in i. consisting of at least 300 bp.

Parts or segments of nucleic acid sequences set forth in ii. consist of at least 300 bp, preferably at least 400 bp, more preferably at least 450 bp, most preferably at least 500 bp of the nucleic acid sequences. In a further preferred embodiment, those parts are selected from SEQ ID NO:1, preferably from 732 bp to 5881 bp of SEQ ID NO:1 e.g. from 2236 bp to 2870 bp.

Furthermore, the present invention encompasses a selection marker comprising a nucleic acid sequence encoding a polyketide synthetase or a polyketide synthetase fragment, wherein said nucleic acid sequence comprises
i. a nucleic acid sequence shown in SEQ ID NO:3, SEQ ID NO:4 or SEQ ID NO:5; or
ii. a nucleic acid sequence which, owing to the degeneracy of the genetic code, can be deduced from the amino acid sequence shown in SEQ ID NO:6 by back translation; or
iii. a functional equivalent of the nucleic acid sequence set forth in i) which is encoded by an amino acid sequence that has at least an identity of 50% with the SEQ ID NO:6; or
iv. parts of the nucleic acid sequence as defined in i., ii. or iii. consisting of at least 300 bp.
v. parts of the nucleic acid sequence as defined in i., ii. or iii. consisting of at least 300 bp comprising
   a) a nucleic acid sequence shown in SEQ ID NO:7; or
   b) a nucleic acid sequence which, owing to the degeneracy of the genetic code, can be deduced from the amino acid sequence shown in SEQ ID NO:8 by back translation; or
   c) a functional equivalent of a nucleic acid sequence set forth in a) which is encoded by an amino acid sequence that has at least an identity of 85% with the SEQ ID NO:8.

Parts or segments of nucleic acid sequences set forth in iii. or v. consist of at least 300 bp, preferably at least 400 bp, more preferably at least 450 bp, most preferably at least 500 bp of the nucleic acid sequences. Preferably, the aforementioned parts or segments of nucleic acid sequences are those set forth in v.a), v.b) or v.c), more preferably those set forth in v.a) or v.b) and most preferably those set forth in v.a). For example, those parts can be selected from 2234 bp to 2865 bp of SEQ ID NO:3.

The functional equivalents of the nucleic acid sequence set forth in iv. are encoded by an amino acid sequence that has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:6.

The functional equivalents of the nucleic acid sequence set forth in v.c) are encoded by an amino acid sequence that has at least an identity of 85%, 86%, 87% or 88% or preferably of 89%, 90%, 91%, 92% or 93% more preferably of 94%, 95% or 96% most preferably of 97%, 98% or 99% with SEQ ID NO:8.

Preferred are nucleic acid sequences as defined above originating from filamentous fungi, preferably phytopathogenic filamentous fungi selected from the group consisting of the genera Neurospora, Alternaria, Podosphaera, Sclerotinia, Physalospora, Botrytis, Corynespora; Colletotrichum; Diplocarpon; Elsinoe; Diaporthe; Sphaerotheca; Cinula, Cercospora; Erysiphe; Sphaerotheca; Leveillula; Mycosphaerella; Phyllactinia; Gloesporium; Gymnosporangium, Leptotthrydium, Podosphaera; Gloedes; Cladosporium; Phomopsis; Phytopora; Phytophthora; Erysiphe; Fusarium; Verticillium; Glomerella; Drechslera; Bipolaris; Personospora; Phaeoisariopsis; Spaceloma; Pseudocercosporella; Pseudoperonospora; Puccinia; Typhula; Pyricularia; Rhizoctonia; Stachosporium; Uncinula; Ustilago; Gaeumannomyces and Fusarium, more preferred from the group consisting of the genera and species Alternaria, Podosphaera, Sclerotinia, Physalospora such as Physalospora canker, Botrytis species such as Botrytis cinerea, Corynespora such as Corynespora melonis; Colletotrichum; Diplocarpon such as Diplocarpon rosae; Elsinoe such as Elsinoe fawcetti, Diaporthe such as Diaporthe citri; Sphaerotheca; Cinula such as Cinula neccata, Cercospora; Erysiphe such as Erysiphe cichoracearum and Erysiphe graminis; Sphaerotheca such as Sphaerotheca fuliginea; Leveillula such as Leveillula taurica; Mycosphaerella; Phyllactinia such as Phyllactinia kakicola; Gloesporium such as Gloesporium kaki; Gymnosporangium such as Gymnosporangium yamadae, Leptotthrydium such as Leptotthrydium pomi, Podosphaera such as Podosphaera leucotricha; Gloedes such as Gloedes pomigena; Cladosporium such as Cladosporium carpophilum; Phomopsis; Phytopora; Phytophthora such as Phytophthora infestans; Verticillium; Glomerella such as Glomerella cingulata; Drechslera; Bipolaris; Personospora; Phaeoisariopsis such as Phaeoisariopsis vitis; Spaceloma such as Spaceloma ampelina; Pseudocercosporella such as Pseudocercosporella herpotrichoides; Pseudoperonospora; Puccinia; Typhula; Pyricularia such as Pyricularia oryzae; Rhizoctonia; Stachosporium such as Stachosporium nodorum; Uncinula such as Uncinula necator; Ustilago; Gaeumannomyces species such as Gaeumannomyces graminis and Fusarium such as Fusarium dimerium, Fusarium merismoides, Fusarium lateritium, Fusarium decemcellulare, Fusarium poae, Fusarium tricinctum, Fusarium sporotrichioides, Fusarium chlamydosporum, Fusarium moniliforme, Fusarium proliferatum, Fusarium anthophilum, Fusarium subglutinans, Fusarium nygamai, Fusarium oxysporum, Fusarium solani, Fusarium culmorum, Fusarium sambucinum, Fusarium crookwellense, Fusarium avenaceum ssp. avenaceum, Fusarium avenaceum ssp. aywerte, Fusarium avenaceum ssp. nurragi, Fusarium hetrosporum, Fusarium acuminatum ssp. acuminatum, Fusarium acuminatum ssp. armeniacum, Fusarium longipes, Fusarium compactum, Fusarium equiseti, Fusarium scripi, Fusarium polyphialidicum, Fusarium semitectum and Fusarium beomiforme and especially preferred from the genera Fusarium such as Fusarium graminearum, most preferred from the group consisting of the genera and species Fusarium, Fusarium dimerium, Fusarium merismoides, Fusarium lateritium, Fusarium decemcellulare, Fusarium poae, Fusarium tricinctum, Fusarium sporotrichioides, Fusarium chlamydosporum, Fusarium moniliforme, Fusarium proliferatum, Fusarium anthophilum, Fusarium subglutinans, Fusarium nygamai, Fusarium oxysporum, Fusarium solani, Fusarium culmorum, Fusarium sambucinum, Fusarium crookwellense, Fusarium avenaceum ssp. avenaceum, Fusarium avenaceum ssp. aywerte, Fusarium avenaceum ssp. nurragi, Fusarium hetrosporum, Fusarium acuminatum. ssp. acuminatum, Fusarium acuminatum ssp. armeniacum, Fusarium longipes, Fusarium compactum, Fusarium equiseti, Fusarium scripi, Fusarium polyphialidicum, Fusarium semitectum and Fusarium beomiforme, wherein Fusarium graminearum is most preferred.

Preferred non-phytopathogenic filamentous fungi are fungi of the group consisting of the genera Neurospora such as Neurospora crassa, Aspergillus such as Aspergillus parasiticus, Aspergillus nidulans, Aspergillus niger and Wangiella such as Wangiella dermatidis.

The term “comprising” means that the nucleic acid sequence according to the invention can be flanked by additional nucleic acid sequences that have on the 5′ end a sequence length of at least 1000 bp and preferably at least 500 bp, more preferably at least 100 bp, most preferably at least 50 bp and on the 3′ a sequence length of at least 1000 bp and preferably at least 500 bp, more preferably at least 100 bp and most preferably at least 50 bp.

“Functional equivalents” in the present context describe nucleic acid sequences which hybridize under standard conditions with the nucleic acid sequence or portions of the nucleic acid sequence having the function of the a selection marker.

It is advantageous to use short oligonucleotides of a length of 10-50 bp, preferably 15-40 bp, for example of the conserved or other regions, which can be determined via comparisons with other related genes in a manner known to the skilled worker for the hybridization. Alternatively, it is also possible to use longer fragments of the nucleic acids according to the invention or the complete sequences for the hybridization. These standard conditions vary depending on the nucleic acid used, viz. oligonucleotide, longer fragment or complete sequence, or depending on which type of nucleic acid, viz. DNA or RNA, is being used for the hybridization. Thus, for example, the melting temperatures for DNA:DNA hybrids are approx. 10° C. lower than those of DNA:RNA hybrids of equal length.

Standard conditions are understood to mean, depending on the nucleic acid, for example temperatures between 42 and 58° C. in an aqueous buffer solution with a concentration of between 0.1 and 5×SSC (1×SSC=0.15 M NaCl, 15 mM sodium citrate, pH 7.2) or additionally in the presence of 50% formamide such as, for example, 42° C. in 5×SSC, 50% formamide. The hybridization conditions for DNA:DNA hybrids are advantageously 0.1×SSC and temperatures of between approximately 20° C. and 45° C., preferably between approximately 30° C. and 45° C. The hybridization conditions for DNA:RNA hybrids are advantageously 0.1×SSC and temperatures of between approximately 30° C. and 55° C., preferably between approximately 45° C. and 55° C. These temperatures stated for the hybridization are melting temperature values which have been calculated by way of example for a nucleic acid with a length of approx. 100 nucleotides and a G+C content of 50% in the absence of formamide. The experimental conditions for DNA hybridization are described in specialist textbooks of genetics such as, for example, Sambrook et al., “Molecular Cloning”, Cold Spring Harbor Laboratory, 1989 and can be calculated using formulae known to the skilled worker, for example as a function of the length of the nucleic acids, the type of the hybrids or the G+C content. The skilled worker can find more information on hybridization in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

A functional equivalent is furthermore also understood to mean, in particular, natural or artificial mutations of the relevant nucleic acid sequences of the polyketide synthetase (PKS) as set forth in SEQ ID NO:1, SEQ ID NO:2, SEQ ID NO:3, SEQ ID NO:4 or SEQ ID NO:5 and its homologs from other organisms, wherein mutations comprise substitutions, additions, deletions, inversions or insertions of one or more nucleotide residues. This may also lead to a modification of the corresponding amino acid sequence of the PKS by substitution, insertion or deletion of one or more amino acids.

Thus, the scope of the present invention also extends to, for example, those nucleotide sequences which are obtained by modification of the nucleic acid sequence of the selection marker described by SEQ ID NO:1 or by SEQ ID NO:2 or SEQ ID NO:3, SEQ ID NO:4, SEQ ID NO:5 respectively. The purpose of such a modification can be, for example, the insertion of further cleavage sites for restriction enzymes, the removal of excess DNA, or the addition of further sequences. Said nucleic acid sequences should still maintain the desired function as marker for targeted transformation, despite the deviating nucleic acid sequence.

The term “identity” or “homology” between two nucleic acid sequences or polypeptide sequences is defined by the identity of the nucleic acid sequence/polypeptide sequence by in each case the entire sequence length, which is calculated by alignment with the aid of the program algorithm GAP (Wisconsin Package Version 10.0, University of Wisconsin, Genetics Computer Group (GCG), Madison, USA), setting the following parameters:

| | |
|---|---|
| Gap Weight: 8 | Length Weight: 4 |
| Average Match: 2,912 | Average Mismatch: −2,003 |

The term homology when used herein is the same as the term identity.

Functional equivalents thus encompass naturally occurring variants of the sequences described herein, and also artificial, for example chemically synthesized, nucleic acid sequences adapted to the codon usage, or the amino acid sequences derived therefrom.

Moreover, SEQ ID NO:1, SEQ ID NO:2, SEQ ID NO:3, SEQ ID NO:4 or SEQ ID NO:5, nucleic acid sequences derived from the amino acid sequence SEQ ID NO:6 by back translation or parts of the aforementioned nucleic acid sequences can be used for the detection and isolation of functional equivalents of other fungi on the basis of sequence identities. In this context, part or all of the sequence of the SEQ ID NO:1, SEQ ID NO:2, SEQ ID NO:3, SEQ ID NO:4 or SEQ ID NO:5 or nucleic acid sequences derived from the amino acid sequence SEQ ID NO:6 by back translation can be used as probe (e.g. hybridization probe) for screening in a genomic library or a cDNA library of the fungal species in question or in a computer search for sequences of functional equivalents in electronic databases. Especially for computer search for sequences of functional equivalents in electronic databases, the amino acid sequence SEQ ID NO:6 or parts of the amino acid sequence SEQ ID NO:6 are useful.

For the preparation of hybridization probes, SEQ ID NO:1, SEQ ID NO:2, SEQ ID NO:3, SEQ ID NO:4 or SEQ ID NO:5 or parts of the aforementioned nucleic acid sequences can be used. The preparation of these probes and the experimental procedure are known. For example, this can be effected via the tailor-made preparation of radioactive or nonradioactive probes by means of PCR and the use of suitably labeled oligonucleotides, followed by hybridization experiments. The technologies required for this purpose are given, for example, in T. Maniatis, E. F. Fritsch and J. Sambrook, “Molecular Cloning: A Laboratory Manual”, Cold Spring Harbor Laboratory, Cold Spring Harbor, N.Y. (1989). The probes in question can furthermore be modified by standard technology (lit. SDM or random mutagenesis) in such a way that they can be employed for other purposes, for example as probe which hybridizes specifically with mRNA and the corresponding coding sequences in order to analyze the corresponding sequences in other organisms.

Furthermore, the cDNA could be used to engineer recombinant microorganisms to produce polyketide agents of pharmaceutical or agricultural interest as described by Pfeifer et al. (Pfeifer B A, Admiraal S J, Gramajo H, Cane D E, Khosla C., Science 2001 Mar. 2;291(5509):1790-2). Thus, the present invention also comprises polypeptides with the biological acitivity of a polyketide synthetase encoded by a nucleic acid sequence comprising
i. a nucleic acid sequence shown in SEQ ID NO:5 or
ii. a nucleic acid sequence which, owing to the degeneracy of the genetic code, can be deduced from the amino acid sequence shown in SEQ ID NO:6 by back translation; or
iii. nucleic acid sequence which is encoded by a functional analogue of an amino acid sequence that has at least an identity of 50% with the SEQ ID NO:6.

The term “functional analogues” describes nucleic acid sequences which are capable of bringing about the expression, in a filamentous fungi, of a polypeptide with the biological activity of polyketide synthetase and which can be deduced from an amino acid sequence by back translation which has a defiened degree of identity with SEQ ID NO:6. The functional analogues set forth in iii) have at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with the SEQ ID NO:6.

Thus, the present invention also encompasses, for example, those nucleotide sequences which are obtained by modification of the abovementioned nucleic acid sequences. For example, such modifications can be generated by techniques with which the skilled worker is familiar, such as “site directed mutagenesis”, “error prone PCR”, “DNA shuffling” (Nature 370, 1994, pp. 389-391) or “staggered extension process” (Nature Biotechnol. 16, 1998, pp. 258-261). The purpose of such a modification can be, for example, the insertion of further cleavage sites for restriction enzymes, the removal of DNA in order to truncate the sequence, the substitution of nucleotides in order to optimize the codons, or the addition of further sequences. Proteins which are encoded via modified nucleic acid sequences must retain the desired functions despite a deviating nucleic acid sequence.

Functional analogues thus comprise naturally occuring variants of the herein-described sequences and artificial nucleic acid sequences, for example those which have been obtained by chemical synthesis and which are adapted to the codon usage, and also the amino acid sequences derived from them.

As explained above, also the expression cassette or the vector comprising a PKS encoding nucleic acid sequence may comprise at least an additional selection marker, preferably the hygromycin resistance gene so that in a particular preferred embodiment, the selection of the succesfully transformed filamentous fungi can be carried out by hygromycin resistance of successfully transformed clones and by the presence of pigment (color) of successfully transformed clones. Most preferably, the vector comprising the PKS encoding nucleic acid sequence is a vector according to the invention comprising a PKS encoding nucleic acid sequence. In addition to the aforementioned selection method homologous recombination can be confirmed by PCR based on oligonucleotides preferably derived from the vector sequence flanking the 5 and 3 region of the gene to be inserted. Specific examples of these primers are given in the examples.

The invention furthermore relates to the use of polyketide synthetase encoding nucleic acid sequences as marker for targeted transformation in filamentous fungi.

Preferably, the present invention comprises the use of a nucleic acid sequence comprising
a) a nucleic acid sequence encoding a polyketide synthetase; or
b) parts of the nucleic acid sequence as defined in i., ii. or iii. consisting of at least 300 bp
   for transformation of filamentous fungi.

Preferred is the use of a nucleic acid sequence as marker for targeted transformation in filamentous fungi said nucleic acid comprising
i. a nucleic acid sequence shown in SEQ ID NO:1 or SEQ ID NO:2; or
ii. parts of the nucleic acid sequence as defined in i. consisting of at least 300 bp, preferably at least 400 bp, more preferably at least 450 bp and most preferably at least 500 bp;

Equally preferred is the use of a nucleic acid sequence as marker for targeted transformation in filamentous fungi, said nucleic acid comprising
iii. a nucleic acid sequence shown in SEQ ID NO:3, SEQ ID NO:4 or SEQ ID NO:5; or
iv. a nucleic acid sequence which, owing to the degeneracy of the genetic code, can be deduced from the amino acid sequence shown in SEQ ID NO:6 by back translation; or
v. a functional equivalent of the nucleic acid sequence set forth in i) or iii) which is encoded by an amino acid sequence that has at least an identity of 40% with the SEQ ID NO:6; or
vi. a nucleic acid sequence shown in SEQ ID NO:9 or SEQ ID NO: 11;
vii. a nucleic acid sequence which, owing to the degeneracy of the genetic code, can be deduced from the amino acid sequence shown in SEQ ID NO:10, SEQ ID NO:12 or SEQ ID NO:13 by back translation; or
viii. parts of the nucleic acid sequence as defined in iii., iv, v., vi or vii. consisting of at least 300 bp, preferably at least 400 bp, more preferably at least 450 bp and most preferably at least 500 bp; or
ix. parts of the nucleic acid sequence as defined in iii., iv, v., vi or vii. consisting of at least 300 bp, preferably at least 400 bp, more preferably at least 450 bp, and most preferably at least 500 bp comprising
   a) a nucleic acid sequence shown in SEQ ID NO:7; or
   b) a nucleic acid sequence which, owing to the degeneracy of the genetic code, can be deduced from the amino acid sequence shown in SEQ ID NO:8 by back translation; or
   c) a functional equivalent of a nucleic acid sequence set forth in a) which is encoded by an amino acid sequence that has at least an identity of 68% with the SEQ ID NO:8.

The nucleic acid sequences according to i. to ix encode for a polypeptide with the biological function of a polyketide synthetase or for a fragment of the aforementioned polypeptide.

Under the aforementioned sequences, the nucleic acid sequences according to i., ii., iii., iv., v. as well as parts of the aforementioned nucleic acid sequence consisting of at least 300 bp, preferably at least 400 bp, more preferably at least 450 bp, most preferably at least 500 bp are preferred. Those parts are preferably those set forth in ix.

Preferred phytopathogenic and non-phytopathogenic filamentous fungi are those mentioned above. The aforementioned nucleic acid sequences are hereinbelow also termed “PKS marker”. Preferably, the term “PKS marker” designates nucleic acid sequences according to i., ii., iii., iv., v. as well as parts of the aforementioned nucleic acid sequence consisting of at least 300 bp, preferably at least 400 bp, more preferably at least 450 bp, and most preferably at least 500 bp are preferred.

The functional equivalents of the nucleic acid sequence set forth in iv. can be deduced from a functional equivalent of the amino acid sequence shown in SEQ ID NO:6 by back translation having at least an identity of 40%, 41%, 42%, 43%, 44%, 45%, 46%, 47%, 48% or 49% preferably of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% and most preferably of 91%, 92%, 93%, 94%, 95%, 96%, 97%, 98% or 99% with the SEQ ID NO:6.

The functional equivalents of the nucleic acid sequence set forth in ix.c) can be deduced from a functional equivalent of the amino acid sequence shown in SEQ ID NO:8 by back translation having at least an identity of 68%, 69%, 70%, 71%, 72%, 73%, 74%, 75%, 76%, 77% or 78% preferably of 79%, 80%, 81%, 82%, 83%, 84% or 85% more preferably of 86%, 87%, 88%, 89% or 90% most preferably of 91%, 92%, 93%, 94%, 95%, 96%, 97%, 98% or 99% with the SEQ ID NO:8.

The use of a PKS marker for targeted transformation of filamentous fungi can be based on significant reduction in the amount of polyketide synthetase which is present in a filamentous fungi. A reduction in the amount the polyketide synthetase means that the amount of polypeptide is reduced via recombinant methods.

Reduction via recombinant methods can involve “antisense techniques”, which describes a technology for the suppression (reduction) of expression of polyketide synthetase, where a PKS marker is transformed into the respective filamentous fungi in “antisense” orientation under the control of a suitable promoter. This method is used preferably for Aspergillus species, and more preferrably for Aspergillus nidulans. The technologies required herefore are well known by the skilled artisan (for example see Bautista et al., Appl. Environ. Microbiol. 2000; 66(10) 4579-81). Suitable vectors therefore comprise an expression cassette comprising
a) a promotor sequence in functional linkage with a PKS marker in antisense orientation; and optionally
b) further genetic control sequences functionally linked to a nucleic acid sequence according to a).

The afore-mentioned expression cassette is hereinbelow termed as “PKS Marker expression cassette”.

The term “expression cassette” can be defined as follows: An expression cassette comprises a nucleic acid sequence which should be expressed, linked functionally to at least one genetic control element, such as a promoter, and, advantageously, a further control element, such as a terminator. Examples of suitable promotors and terminators are given above. The nucleic acid sequence of the expression casette can be, for example, a genomic or complementary DNA sequence or an RNA sequence, and the semisynthetic or fully synthetic analogs thereof. These sequences can exist in linear or circular form, extrachromosomally or integrated into the genome. The nucleic acid sequences in question can be synthesized or obtained naturally or comprise a mixture of synthetic and natural DNA components, and consist of a variety of heterologous gene segments from various organisms.

Artificial nucleic acid sequences are also suitable in this context as long as they make possible the expression, in a cell or organism, of a polypeptide encoded by a nucleic acid sequence according to the invention and having the biological activity of a polyketide synthetase. For example, synthetic nucleotide sequences can be generated which have been optimized with regard to the codon usage of the organisms to be transformed.

All of the abovementioned nucleotide sequences can be generated from the nucleotide units by chemical synthesis in the manner known per se, for example by fragment condensation of individual, overlapping complementary nucleotide units of the double helix. Oligonucleotides can be synthesized chemically for example in the manner known per se using the phosphoamidite method (Voet, Voet, 2nd Edition, Wiley Press New York, pp. 896-897). When preparing an expression cassette, various DNA fragments can be manipulated in such a way that a nucleotide sequence with the correct direction of reading and the correct reading frame is obtained. The nucleic acid fragments are linked to each other via general cloning techniques as are described, for example, in T. Maniatis, E. F. Fritsch and J. Sambrook, “Molecular Cloning: A Laboratory Manual”, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., “Current Protocols in Molecular Biology”, Greene Publishing Assoc. and Wiley-Interscience (1994).

The term “genetic control element” describes sequences which have an effect on the transcription and, if appropriate, translation of the nucleic acids according to the invention in prokaryotic or eukaryotic organisms. Examples are terminators. Examples of suitable terminators are given above. In addition to the afore-mentioned control sequences, or instead of these sequences, the natural regulation of these sequences may still be present before the actual structural genes and may, if appropriate, have been modified genetically in such a way that the natural regulation has been switched off and the expression of the target gene has been modified, that is to say increased or reduced. The choice of the control sequence depends on the host organism or starting organism. Genetic control sequences furthermore also comprise the 5′-untranslated region, introns or the noncoding 3′ region of genes. Control sequences are furthermore understood as meaning those which make possible a homologous recombination or insertion into the genome of a host organism or which permit the removal from the genome. Genetic control sequences also comprise further promoters, promoter elements or minimal promoters.

The transcription of the PKS marker leads to suppression of the transcription of the natural polyketide synnthethase gene, which can be detected by loss of color of the transformed fungi relative to the respective wild-type strain.

In a preferred embodiment, the reduction via recombinant methods is based on a gene knock out of the polyketide synthethase gene using either an expression cassette additionally comprising the PKS marker or a vector comprising the PKS marker in the respective filamentous fungi. Disruption of the PKS marker will lead to a loss of color.

Preferred phytopathogenic filamentous fungi are those mentioned above. Preferred non-phytopathogenic filamentous fungi are fungi of the group consisting of the genera Aspergillus such as Aspergillus parasiticus, Aspergillus nidulans and Wangiella such as Wangiella dermatidis.

In this connection, the selection of the functional equivalent for the use as marker gene depends on the fungi to be transformed. By preference, the polyketide synthetase fragment has an identity of at least 80%, preferably at least 81%, 82%, 83%, 84%, 85%, 86%, 87%, 88%, 89%, 90%, and especially prefer-ably at least 91%, 92%, 93%, 94%, 95%, 96%, 97%, 98%, 99% identity with the polyketide synthetase of the fungi to be transformed.

For example, for transformation of Fusarium graminearum, a nucleic acid sequence can be selected comprising
i. a nucleic acid sequence shown in SEQ ID NO:1, SEQ ID NO:2, SEQ ID NO:3, SEQ ID NO:4 or SEQ ID NO:5; or
ii. a nucleic acid sequence that has at least an identity of 80% SEQ ID NO:1, SEQ ID NO:2, SEQ ID NO:3, SEQ ID NO:4 or SEQ ID NO:5; or
iii. parts of the nucleic acid sequence as defined in i. or ii. consisting of at least 300 bp, preferably at least 400 bp, more preferably at least 450 bp, and most preferably at least 500 bp.
iv. parts of the nucleic acid sequence as defined in i. or ii. consisting of at least 300 bp, preferably at least 400 bp, more preferably at least 450 bp, and most preferably at least 500 bp comprising
   a) a nucleic acid sequence shown in SEQ ID NO:7; or
   c) a nucleic acid sequence that has at least an identity of 80% with the SEQ ID NO:8.

As mentioned above, another embodiment of the present invention is plasmid vectors for targeted transformation of filamentous fungi comprising a PKS marker. These plasmid vectors are either vectors currently used for targeted transformation of filamentous fungi e.g. such as pAN7 (Punt et al, 1987 Gene 36:117-124) and other vectors that are well known by the skilled artisan or plasmid vectors according to the invention, preferably plasmid vectors according to the invention.

All of the above-mentioned vectors comprising the PKS marker are hereinbelow termed as “PKS vectors”.

A PKS vector is also a vector, which comprises a PKS Marker-expression cassette.

All vectors according to the invention not comprising the PKS marker are hereinbelow termed as “non-PKS vectors”.

The present invention furthermore encompasses a method for preparing mutated filamentous fungi, comprising the steps of transferring a non-PKS vector or a PKS vector into a filamentous fungi; and selecting clones of said filamentous fungi, which contain at least one genetic marker introduced by said plasmid vector.

The term filamentous fungi as well as preferred fungi for this purpose are those mentioned and defined above.

In a preferred embodiment, the method for preparing mutated filamentous fungi, comprising the following steps
a) transferring a PKS vector into a filamentous fungi; and
b) selecting successfully transformed filamentous fungi by the absence of color (pigment).

As explained above, the absence of color is based on significant reduction in the amount of polyketide synthetase (or in the polyketide syntethase activity or instability of polyketide syntethase mRNA, which is present in a filamentous fungi). The absence of color can be monitored for example by comparing the transformed fungi with the respective wild-type fungi of the same species.

If a PKS vector is transferred into a filamentous fungi, the disruption of the PKS gene leads to a loss of color (pigment) whereby the degree of transformation can be determined easily. Resulting transformants are white in contrast to the colored wild-type. Thus, the selection according to step b) is done by monitoring the absence of color (pigment) in the filamentous fungi. In a preferred embodiment, the absence of pigment is monitored by optical means.

Alternatively, the absence of color results from the reduction of the polyketide synthethase via antisense techniques. The absence of color hereby means a “reduction of color” or, preferably, loss of color. Absence of color means a reduction in color of at least 20%, preferably between 20 and 40%, more preferably between 40 and 60%, especially preferaby between 60 and 80% and most preferably between 80% and 100%.

In a more preferred embodiment, the PKS vector comprises at least an additional selection marker, preferably the hygromycin resistance gene. In a particular preferred embodiment, the selection of the successfully transformed filamentous fungi comprising a PKS vector can be carried out by hygromycin resistance of success-fully transformed clones and by the absence of pigment of successfully transformed clones. Most preferably, the PKS vector is a vector according to the invention additionally comprising a PKS marker.

In a further embodiment of the invention, the selection of the succesfully transformed filamentous fungi comprising a non-PKS vector can be carried out by hygromycin resistance of successfully transformed clones.

If a non-PKS vector is used, the vector is linearized by a restriction enzyme cutting in the nucleic acid sequence region of element d). Also nucleic acid sequences exceeding 2000 bp can be used what can be disadvantageous as mentioned above. If a PKS vector is used, the plasmid vector is transferred into a filamentous fungi with the proviso that said vector is linearized by a restriction enzyme in PKS marker nucleic acid sequence. Unlike the non-PKS vectors, the nucleic acid sequence to be expressed recombinantly can also be smaller than 400 bp.

In addition to the aforementioned selection methods set forth in step a) to c), homologous recombination can be confirmed by PCR based on oligonucleotides preferably derived from the vector sequence flanking the 5′ and 3′ regions of the gene to be inserted. Specific examples of these primers are given in the examples.

The plasmid vector may be transferred into the filamentous fungi to be transformed by methods familiar to the skilled worker, preferably via protoplast preparation with driselase or driselase and glucanase as lytic enzyme.

The above-mentioned transformation methods can be also realized in a high throughput screening. Using high throughput screening, many different clones are obtained in parallel so that large numbers of successfully tranformed clones of filamentous fungi can be quickly screened.

The term filamentous fungi as well as preferred fungi for this purpose are those mentioned and defined above.

Due to the convenience of the vector, the above-mentioned KO-plasmid preparation, fungi transformation and screening of the mutants can be at least partially automated so that the whole procedure can also be realized in a high throughput screening. Using high throughput system for example for KO-plasmid preparation and DNA amplification by PCR to screen the recombinant mutants, many different clones are obtained in parallel so that large numbers of transformants can be quickly screened.

Mutagenized filamentous fungi, obtainable according to a method mentioned above, are further encompassed by the present invention.

In an alternative embodiment, the method of transforming filamentous fungi based on the use of polyketide synthetase as marker for transformation comprises the following steps:
a) providing a filamentous fungi characterized by the absence of color (pigment), in which the polyketide synthetase gene is modified in such a way that the polyketide synthetase cannot be functionally expressed;
b) transforming the filamentous fungi of step a) with a “sense expression cassette” or a vector comprising the aforementioned expression cassette;
c) selecting successfully transformed filamentous fungi by the presence of pigment (color).

The nucleic acid sequence as defined in b) i to v. is herein below termed as PKS encoding sequence.

The terms “expression cassette” and “genetic control elements” are explained above.

The “sense-expression cassette” set forth in step b) of the above-mentioned method comprises
a) a promotor sequence in functional linkage with a nucleic acid sequence comprising
   i. a nucleic acid sequence shown in SEQ ID NO:3, 4 or 5; or
   ii. a nucleic acid sequence which, owing to the degeneracy of the genetic code, can be deduced from the amino acid sequence shown in SEQ ID NO:6 by back translation; or
   iii. a functional equivalent of the nucleic acid sequence set forth in i) which is encoded by an amino acid sequence shown in SEQ ID NO:6 that has at least an identity of 40% with the SEQ ID NO:6; or
   iv. a nucleic acid sequence shown in SEQ ID NO:9 or SEQ ID NO: 11;
   v. a nucleic acid sequence which, owing to the degeneracy of the genetic code, can be deduced from the amino acid sequence shown in SEQ ID NO:10, SEQ ID NO:12 or SEQ ID NO:13 by back translation;
   and optionally
b) further genetic control sequences in functionally linked with a nucleic acid sequence according to a).

The expression cassette or vector comprises preferably a polyketide synthetase encoding nucleic acid sequence as set forth in b) i., ii. or iii.

Preferred phytopathogenic filamentous fungi are those mentioned above. Preferred non-phytopathogenic filamentous fungi are fungi of the group consisting of the genera Aspergillus such as Aspergillus parasiticus, Aspergillus nidulans and Wangiella such as Wangiella dermatidis.

The modification of the polyketide synthetase encoding sequence of the respective fungi can be done either by introduction of at least one mutation in the gene encoding a polyketide synthetase or disruption of the gene encoding a polyketide synthetase.

The term “disruption of the PKS marker” means that the PKS marker sequence is disrupted by introducing DNA comprising stop-codons in the PKS marker sequence e.g. by homologous recombination. The respective methods are well known by the skilled artisan.

The term “mutations” of nucleic acid sequences comprises substitutions, additions, deletions, inversions or insertions of one or more nucleotide residues, which have to bring about termination of translation of the corresponding amino acid sequence of the target protein by the substitution, insertion or deletion of one or more amino acids (e.g. a by frame-shift or introduction of stop codon or amendment of nucleic acid sequence). The respective methods are well known by the skilled artisan.

For example, the mutations are carried out in the flanking regions of exon and intron of a PKS gene. These regions can be determined easily by the skilled artisan. For example, in SEQ ID NO:3 the flanking regions between exon are at bp 1022/1023; bp 1067/1068, bp 1361/1362; bp 1067/1068; bp 1361/1362; bp 1067/1068; bp 1361/1362; bp 1416/1417; bp 2399/2400; bp 2447/2448; bp 2675/2676; bp 2738/2739; bp 5744/5745; bp 5792/5793; and/or bp 7205/7206 (Ende 6. exon bp 7205).

The term “functional analogues” is defined above describe, in the present context nucleic acid sequences which are capable of bringing about the expression, in a filamentous fungi, of a polypeptide with the biological activity of polyketide synthetase and which can be deduced from an amino acid sequence by back translation which has at least an identity of 40%, 41%, 42%, 43%, 44%, 45%, 46%, 47%, 48% or 49%, preferably of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78% or 79%, more preferably of 80%, 81%, 82%, 83%, 84%, 85%, 86%, 87%, 88%, 89%, or 90% most preferred of 91%, 92%, 93%, 94%, 95%, 96%, 97%, 98% or 99% with the SEQ ID NO:6.

As explained above, the plasmid vector may be transferred into the filamentous fungi to be transformed by methods familiar to the skilled worker, preferably via protoplast preparation with driselase or driselase and glucanase as lytic enzyme.

The above-mentioned transformation methods can also be realized in a high throughput screening. Using high throughput screening, many different clones are obtained in parallel so that large numbers of successfully tranformed clones of filamentous fungi can be quickly screened.

The invention is now illustrated by the examples which follow, but is not limited thereto.

### EXAMPLES

The recombinant methods on which the exemplary embodiments which follow are based are now described briefly:

A: General Methods

Cloning methods such as, for example, restriction cleavages, DNA isolation, agarose gel electrophoresis, purification of DNA fragments, transfer of nucleic acids to nitrocellulose and nylon membranes, linking of DNA fragments, transformation of E. coli cells, bacterial cultures, sequence analysis of recombinant DNA and Southern and Western Blots were carried out as described by Sambrook et al., Cold Spring Harbor Laboratory Press (1989) and Ausubel, F. M. et al., Current Protocols in Molecular Biology, Greene Publishing Assoc. and Wiley-Interscience (1994); ISBN 0-87969-309-6.

The bacterial strains used hereinbelow (E. coli DH5 or XL1 blue) were obtained from Life Technologies or Stratagene. The vectors were used for cloning. DSM:4527 can be used as F. Graminearum wild-type strain 8/1. Restriction maps of the vectors pUCmini-Hyg and PUCmini-Hyg TA are given in FIGS. 1 and 2.

B: Sequence Analysis of Recombinant DNA

Recombinant DNA molecules were sequenced using an ABI laser fluorescence DNA sequencer following the method of Sanger (Sanger et al., Proc. Natl. Acad. Sci. USA, 74, 5463-5467(1977)). Fragments resulting from a polymerase chain reaction were sequenced and verified in order to avoid polymerase errors in constructs to be expressed.

C: Materials Used

Unless otherwise specified in the text, all of the chemicals used were obtained in analytical grade quality from Fluka (Neu-Ulm), Merck (Darmstadt), Roth (Karlsruhe), Serva (Heidelberg) and Sigma (Deisenhofen). Solutions were prepared using pure pyrogen-free water, referred to in the following text as H₂O, from a Milli-Q water system purification unit (Millipore, Eschborn). Restriction enzymes, DNA-modifying enzymes and molecular-biological kits were obtained from AGS (Heidelberg), Amersham (Brunswick), Biometra (Göttingen), Roche (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Heidelberg). Unless otherwise specified, they were used following the manufacturer's instructions.

All of the media and buffers used for the genetic engineering experiments were sterilized either by filter sterilization or by heating in an autoclave.

In degenerated primer sequences, the following abbreviations are used:

A or T=“W”;

G or C=“s”;

T or C=“Y”;

A or C=“M”;

A or G=“R”;

### EXAMPLES

### Example 1

### Construction of pUCmini-Hyg and PUCmini-Hyg TA Vector

A 2536 bp DNA fragment corresponding to the promoter of glycerol-3-phosphate dehydrogenase (GPD1) from Cochliobolus heterotrophus associated to the hygromycine B resistance gene from Escherichia coli was amplified by PCR with the oligonucleotides

(SEQ ID NO 14)

P1 5′ atgaagcttggggtttgagggccaatggaacgaaactagtgtac
cacttgacc 3′;
and

(SEQ ID NO 15)

P2 5′gacagatctggcgccattcgccattcag 3′

using pGUS5 as template (Mönke, E. and Schafer, W., 1993, Mol. Gen. Genet. 241: 73-80). The PCR is done using standard protocols; e.g. as described in Maniatis et al., Mol. Cloning.

The resulting DNA fragment was inserted in the plasmid pFDX3809 (WO 01/38504) by the restriction site Hind III and Bgl II introduced by the oligonucleotides P1 and P2. The resulting plasmid pHygB serves as template for a further PCR, wherein the Oligonucleotides

(SEQ ID NO 16)

ANK 518 5′ ggaatcggtcaatacactac 3′

(SEQ ID NO 17)

ANK 519 5′ tgtagatctctattcctttgccctcggacgagt 3′

are used to shorten the hygromycin B resistance gene specifically. The resulting PCR fragment comprising 575 bp of the 3′end of the hygromycine gene was inserted in the plasmid pHygB via the restriction sites Nde I/Bgl II generating the plasmid pHygB-NOS.

A Hind III/Ssp I DNA fragment of 2019 bp containing the expression cassette GPD1 promoter, the hygromycine B resistance gene and the nopaline synthase terminator was isolated from pHygB-NOS and inserted in the pUCmini plasmid (=plasmid pFDX3809, see WO 01/38509) previously treated with EcoRI and HindIII restriction enzymes to give the plasmid pUCmini-Hyg; to do so, the EcoRI ends were made compatible with Ssp I by a fill-in treatment using the Klenow fragment of DNA polymerase I. A second version of pUCmini-Hyg, called pUCmini-Hyg-TA, was obtained by the insertion of the following adaptor in the NotI/AscI restriction sites of pUCmini-Hyg:

(SEQ ID NO 18)

5′ GGCCGCCACGGATATCTTGGCCAAAGAATTCCTGG 3′

The adaptor contains 2 XcmI restriction sites so that XcmI digest of pUCmini-Hyg-TA creates T-overhangs that permits direct cloning of PCR products made with the classical Taq-polymerases.

### Example 2

### Construction of the PKS Comprising vector “pUCmini-Hyg-PKS”

The nucleic acid sequence encoding PKS was amplified by PCR with degenerated primers

(SEQ ID NO 20)

LC1 5′-GAY CCI MGI TTY TTY AAY ATG-3′

(SEQ ID NO 21)

LC2c 5′-GTI CCI GTI CCR TGC ATY TC-3′

based on the conserved amino acid sequence of the PKS gene sequences from Aspergillus nidulans, Colletotrichum lagenarium, Penicillium patulum, and Aspergillus parasiticus (Bingle et al., 1999) using genomic DNA of Fusarium graminearum as template. Thermal cycling parameters consisted of an initial denaturation at 94° C. for 3 min followed by 34 cycles of 94° C. for 1 min (denaturation), 55° C. for 1 min (annealing), 72° C. for 3 min (extension) and a final extension at 72° C. for 10 min according to standard procedures. The resulting PCR product was cloned into the PGEM-T vector (Promega, Mannheim, Germany) to give the plasmid pGEMT/PKS833 and sequenced. A 633 bp DNA fragment (2236 bp to 2870 bp of SEQ ID NO:1; corresponding to 2234 bp to 2865 bp of SEQ ID NO:3; set forth in SEQ ID NO:18) was amplified by PCR using the oligonucleotides

(SEQ ID NO 22)

ANK593 5′ ATAAGAATGCGGCCGCAATGGCCCTCGAAACAGC 3′

(SEQ ID NO 23)

ANK594 5′ AAATGGCGCGCCGCGCCCAGAATGACACC 3′

and cloned into the plasmid pUCmini-Hyg using the restriction site NotI and AscI present in the oligonucleotide sequences. The resulting plasmid pUCmini-Hyg-PKS is used for homologous recombination.

The flanking regions of the PKS DNA fragment were obtained by inverse PCR (Triglia T, Peterson M G, Kemp D J, Nucleic Acids Res 1988 Aug. 25; 16(16):8186). Genomic DNA was treated with the restriction enzymes PstI, NcoI, or XhoI respectively. DNA was then self-ligated to get circular DNA molecule. The latter was used as template for the inverse PCR reaction using the primers

P1A: 5′ TGCCACCTGTAGTCTGCAATCAG 3′ (SEQ ID NO 24)
and
P2A: 5′ TGACTAACCCTGACAACTTCGCTG 3′ (SEQ ID NO 25)

deduced from the polyketide synthetase (PKS) DNA fragment of the plasmid pGEM-T/PKS833 described above.

In a second step, the PCR product was reamplified with the nested primers

| | |
|---|---|
| P1B:5′ CCAGGATCCGACTGCTCAG 3 | (SEQ ID NO 26) |
| and | |
| P2B:5′ CTACATCGAGATGCACGGCAC 3′ | (SEQ ID NO 27) |

(deduced from the PKS DNA fragment of the plasmid pGEMT/PKS833), cloned into the pPCR-XL-TOPO vector (Invitrogen) and sequenced to get SEQ ID NO:1.
Identification of the Genomic DNA Sequence

The remaining parts of the flanking regions were obtained by Tail-PCR (Liu Y G, Whittier R F; Genomics 1995 Feb. 10; 25(3):674-81) using 9 arbitrary degenerated primers

(SEQ ID NO 28)

FJM-tail-AD1 5′-NGT CGA SWG ANA WGA A-3′,

(SEQ ID NO 29)

FJM-tail-AD2 5′-GTN CGA SWC ANA WGT T-3′,

(SEQ ID NO 30)

FJM-tail-AD3 5′-WGT GNA GWA NCA NAG A-3′,

(SEQ ID NO 31)

FJM-tail-AD4 5′-NTC GAS TWT SGW GTT-3′

(SEQ ID NO 32)

FJM-tai1-AD6 5′-TGW GNA GWA NCA SAG A-3′,

(SEQ ID NO 33)

FJM-tail-AD7 5′-AGW GNA GWA NCA WAG G-3′,

(SEQ ID NO 34)

FJM-tail-AD8 5′-CAW CGI CNG AIA SGA A-3′
and

(SEQ ID NO 35)

FJM-tail-AD9 5′-TCS TIC GNA CIT WGG A-3′

coupled to the primer

(SEQ ID NO 36)

TailPKS1c 5′-TTG TTA CTG GAG AGG TAA TGA AG-3′

specific for the 5′ PKS flanking region deduced from SEQ ID NO:1, or coupled to the primer

(SEQ ID NO 37)

TailPKS2c 5′-TGA GAC AGA TCT CGC GAG CCC TC-3′

specific for the 3′ PKS flanking region deduced from SEQ ID NO:1. After subcloning and subsequent sequencing of the PCR products SEQ ID NO:3 was obtained.
Identification of the cDNA Sequence of Polyketide Synthetase

The PKS cDNA sequence was obtained by RT-PCR with a crude RNA preparation from Fusarium graminearum and various primers deduced from the genomic sequence. This was done according the classicals methods (Ausubel, F. M. et al., Current Protocols in Molecular Biology, Greene Publishing Assoc. and Wiley-Interscience (1994); ISBN 0-87969-309-6). Alignement of cDNA and genomic PKS sequences permits to be identified precisely the location of introns in the genomic sequence.

### Example 3

### Transformation of F. graminearum

50 ml of CM-medium (Leach et al., 1982, J. Gen. Microbiol. 128: 1719-1729) were inoculated with approximately 10⁵ conidia, and incubated for 2 days at 28° C., 140 rpm. Resulting hyphae were homogenized in a Warring-Blender; 200 ml CM were inoculated with 10 ml hyphal suspension, and incubated overnight at 24° C. Mycel were trapped on a sterile filter, and washed two times with sterile water. 2 g of the hyphae were resuspended in 20 ml Driselase/Glucanase (Interspex Products, San Maneo, USA; 5%/3% in 700 mM NaCl, pH 5.6), and digested 2½ to 3 h at 28° C., 75 rpm. Undigested hyphal were removed from the protoplast suspension by filtration through gauze and Nybold membrane (50 μm pore size). The protoplast suspensions were combined with 700 mM NaCl and again passed through the gauze and the Nybold membrane. The protoplasts were pelleted by centrifugation (1300×g) in a swing-out rotor and washed two times with ice-cold NaCl 700 mM and centrifuged (830×g). Then the protoplasts were resuspended in STC (0.8 M sorbitol, 50 mM Tris-HCl pH 8.0, 50 mM CaCl₂) and stored on ice until transformation (maximum 1 week).

For transformation, protoplasts were resuspended in 4 parts STC and 1 part SPTC (0.8 M sorbitol, 40% polyethylene glycol 4000, 50 mM Tris-HCl pH 8.0, 50 mM CaCl₂) at a concentration of 0.5-2×10⁸/ml; 30 μg of the pUCmini-Hyg-PKS plasmid DNA linearized with the Eco47III restriction site inside the PKS fragment and 5 μl heparin (5 mg/ml in STC) were added to 100 μl of the protoplast suspension in 10 ml tubes. After mixing, samples were incubated on ice for 30 min. 1 ml SPTC was mixed with the suspension and incubated at room temperature for 20 min. Protoplasts were mixed gently into 200 ml regeneration medium (0.1% (w/v yeast extract, 0.1% (w/v. caseinhydrolysate, 34.2% (w/v) sucrose, 1.6% (w/v) granulated agar) at 43° C. and spread on 94 mm plates (20 ml per plate). The plates were incubated at 28° C. After 12-24 h, the plates were overlaid with 10 ml per plate water based selective medium (16 g/l granulated agar, 100 mg/l hygromycin and further incubated at 28° C. until transformants were obtained, which were transferred to fresh CM-Hyg-plates (consisting of CM-media, 100 μg/ml hygromycin and 2% (w/v) agar. The transformants were isolated by single spore isolation. For generation of conidia, the transformants were cultivated on SNA plates (Nirenberg, 1981, Canadian J. Botany 59: 1599-1609) under UV-light 7-14 days at 18° C. Dilutions of conidia were plated on CM-Hyg plates, and single colonies were transferred from these plates to fresh CM-Hyg plates.

### Example 4

### Southernblot Analysis

Genomic DNA was isolated from frozen hyphal material using the Puregene Genomic DNA Isolation Kit (Gentra Systems, Minneapolis USA) and digested for 6 h with NruI restriction enzyme. The genomic DNA was separated by electrophoresis on a 1% (w/v) agarose gel and blotted onto a nylon membrane (Hybond NX; Amersham Pharmacia Biotech, Buckinghamshire, England). A digoxigenin labeled probe was generated by PCR based on specific primers PKS forward 5′-GCG CTT GAG ATG GCT AGT ATC G-3′ and and PKS reverse 5′-GTG CCG TGC ATC TCG ATG TAG-3′ using pGEM-T/PKS833 as template and digoxigenin labeled dUTPs by PCR reaction according to the recommendation of the manufacturer (Roche Diagnostics GmbH, Mannheim). PCR conditions were 94° C. for 3 min (initial denaturation) followed by 30 cycles of 94° C. for 30 sec (denaturation), 55° C. for 45 sec (annealing), 72° C. for 1 min (extension) and a final extension at 72° C. for 10 min. The non-radioactive hybridization and the detection were done under highly stringent conditions as described in Roche Molecular Biochemicals DIG Application Manual for Filter Hybridization (Roche Diagnostics GmbH, Mannheim).

To confirm the insertion of the vector construct into the PKS locus in comparison with the wild type gene, primers

(SEQ ID NO 38)

EF-PKS 5′ atgtctccaaaggaagctgagc 3′;
and

(SEQ ID NO 39)

ER-PKS 5′ tcgagtgatggatactgcttcg 3′

are constructed based on the PKS DNA sequence from the plasmid pGEM-T/PKS833; four universal primers are constructed, wherein

(SEQ ID NO 40)

Lac 92 5′ cggctacactagaaggacagtatttggta 3′

(SEQ ID NO 41)

Lac 93 5′ gtcaggcaactatggatgaacgaaatagac 3′

(SEQ ID NO 42)

Lac 94 5′ acccatctcataaataacgtcatgc 3′;
and

(SEQ ID NO 43)

Lac 95 5′ caactctatcagagcttggttga 3′

permit amplification of a 412 bp DNA fragment of the hygromycin cassette.

PCR reactions were conducted in classical conditions: 94° C. for 3 min (initial denaturation) followed by 30 cycles of 94° C. 60 sec (denaturation), 55° C. for 90 sec (annealing), 72° C. for 90 sec (extension) and a final extension at 72° C. for 10 min. Six (6) recombinant clones resistant to hygromycine were analyzed by PCR using the primer set Lac 94/Lac 95 specific for the hygromycin resistance gene. All the mutants were found to present the expected DNA fragment of 412 bp, indicating the integration of the plasmid pUCmini-Hyg-PKS in the genome.

A 712 bp corresponding to the PKS gene could be amplified with the primer set EF-PKS/ER-PKS mentioned above using genomic DNA from a wild type strain; however no PCR fragments were amplified with genomic DNA from the recombinant clones indicating that the PKS gene is disrupted by the insertion of pUCmini-Hyg-PKS. This was confirmed by PCR amplification EF-PKS combined with Lac 93 (hybridizing to the plasmid backbone near Not I restriction site) and ER-PKS combined with Lac 92 (hybridizing to the plasmid backbone near Asc I restriction site). In both cases, DNA fragments of about 600 bp were amplified for the recombinant clones but not for the wild type strain (WT). Alltogether, the PCR analysis using the different primer sets proves that the plasmid pUCmini-Hyg-PKS was targeted specifically in the PKS locus by homologous recombination. This process permits the PKS gene to be disrupted since the recombinant mutants were found to lack the typical pigmentation (purple) of the wildtype strain.

### Example 5

### FUNCTIONAL Expression of Green Fluorescent Protein (GFP) in Fusarium graminearum

A) Plasmid Construction

In a first step, a 67 bp DNA fragment encoding the peptide leader of the first 23 amino acids from N-terminus of the yeast ARH1 (SwissProt;P48360) was amplified by PCR using the primers

Lac 80

(SEQ ID NO 44)

5′ cccgaattcatgagctttgttcaaataagg 3′
and
Lac 81

(SEQ ID NO 45)

5′ ttattctagattttccatgggaatggatacagtcttacg 3′

In a second step, a 734 bp DNA fragment encoding the Green Fluorescent Protein (GFP) was amplified by PCR using the plasmid pEGFP-N2 (Genbank; U57608) and the primers

Lac 84

(SEQ ID NO 46)

5′ cgccaccatggtgagcaagggcgaggagctgtt 3′
and
Lac 85

(SEQ ID NO 47)

5′ tatgatctagagtcgcggccgctttacttgtacagctcg 3′.

The PCR products were assembled in frame with the Nco I restriction sites present in the oligonucleotides Lac 81 and Lac 84 and cloned in the expression plasmid pYes2 (Invitrogen) using the restriction sites EcoRI and Xba I present in the oligonucleotides Lac 80 and Lac 85, respectively. In the resulting plasmid pLAC7, the recombinant gene encoding GFP is under the control of the galactose (Gal 1) promoter and cytochrome C1 terminator.

A 2892 bp DNA fragment containing the GFP expression cassette was isolated from pLac7 using the restriction sites Nae I and Bsa I and cloned in the plasmid pUCmini-Hyg-PKS (see example 2). To do so, pUCmini-Hyg-PKS was firstly cut by Asc I and filled in according to classical method then treated with Bsa I. The resulting plasmid pUCmini-Hyg-PKS-GFP contains all genetic elements permitting the production of recombinant GFP in Fusarium graminearum.

B) Transformation of Fusarium graminearum with pUCmini-Hyg-PKS-GFP and Analysis of Transformants

The transformation was done as described in example 3, wherein pUCmini-Hyg-PKS-GFP was linearized with EcoR47III. The correct integration of the plasmid in the PKS locus was observed after single conidiation by the absence of pigmentation of the recombinant mutants.

In addition, the integration was confirmed by PCR as described in example 4 using the following primer combinations EF-PKS (see example 4; SEQ ID NO:38) and ER-PKS (see example 4; SEQ ID NO:39), whereby no amplification were observed since the gene PKS is disrupted, whereas wild type strain or unspecific mutants presented a 714 bp DNA fragment corresponding to the expected PKS DNA fragment.

Using the primer combination EF-PKS (see example 4; SEQ ID NO:38) and

(SEQ ID NO 48)

Lac 211 5′ gcttctaatccgtactagtggatca 3′

the amplification of a 835 bp DNA corresponding to the 5′ end plasmid integration in the PKS locus of the mutants was observed. No DNA fragment was amplified for the wildtype strain due the absence of the DNA sequence complementary to Lac211.

The Primer Combination

(SEQ ID NO 49)

ANK 458 5′ ctttgatcttttctacggggtctga 3′

and ER-PKS (see example 4; SEQ ID NO:39) led to the amplification of a 718 bp DNA corresponding to the 3′ end plasmid integration in the PKS locus of the mutants. No DNA fragment was amplified for the wildtype strain due the absence of the DNA sequence complementary to ANK 458.

C) Detection of the Production of GFP

The recombinant mutants were grown for a few days in CM-Hyg medium as described in example 3 except for glucose which was replaced by galactose as a carbon source. The fluorescence of GFP was detected using the polarstar spectrophotometer (Firma BMG; Ex: 385 nm and Em: 520 nm). In these conditions fluorescence was observed for the strains which showed integration of the plasmid whereas no fluorescence was observed for the wildtype strains.

### BRIEF DESCRIPTION OF THE FIGURES

FIG. 1: Map of pUCmini-Hyg

FIG. 1: Map of PUCmini-Hyg TA

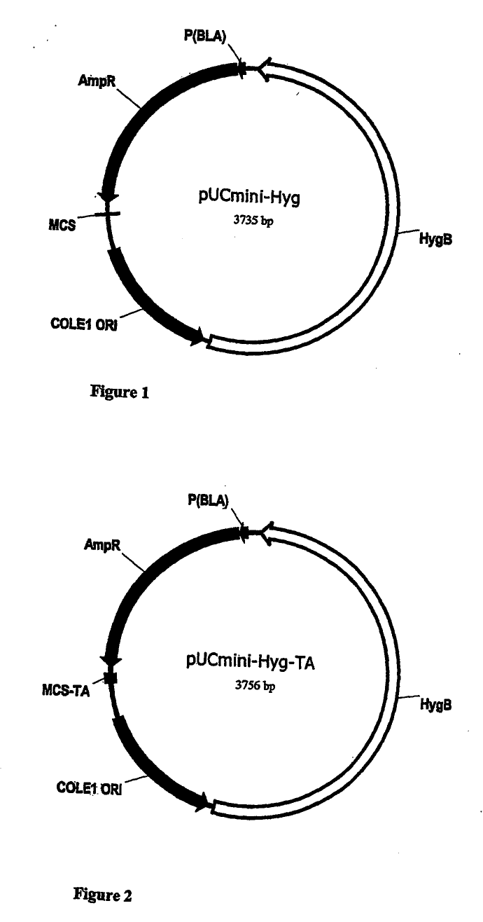

1. A plasmid vector for targeted transformation of filamentous fungi comprising
a) an origin of replication for a host organism not originating from the filamentous fungi to be transformed;
b) a selection marker for a host organism not originating from the filamentous fungi;
c) a promotor facilitating recombinant expression in fungi that is functionally linked to the coding region of the hygromycin resistance gene which is functionally linked to a terminator which facilitates transcription termination in filamentous fungi;
wherein the overall size of the elements a), b) and c) does not exceed 4500 bp; and
d) a nucleic acid sequence which is homologous to nucleic acid sequences of the filamentous fungi to be transformed and makes homologous recombination in the filamentous fungi to be transformed possible.

2. A plasmid vector as claimed in claim 1, wherein the origin of replication a) originates from bacteria.

3. A plasmid vector as claimed in claims 1 to 2, wherein the selection marker b) imparts a resistance to antibiotics.

4. A plasmid vector according to claims 1 to 3, wherein the promotor of element c) is selected from the group consisting of the GPD-1-, PX6-, TEF-, CUP1-, PGK-, GAP1-, TPI, PHO5-, AOX1, GAL10/CYC-1, CYC1, OliC-, ADH-, TDH-, Kex2-, MFa- and the NMT-promotor.

5. A plasmid vector according to claims 1 to 4, wherein the terminator of element c) is selected from the group consisting of the AOX1-, nos-, PGK-, TrpC- and the CYC1-terminator.

6. A plasmid vector according to claims 1 to 5, wherein the promotor of element c) is the GPD-1-promotor and the terminator of element c) is the nos-terminator.

7. A plasmid vector according to claims 1 to 6, wherein the nucleic acid sequence d) is functionally linked to a promotor facilitating recombinant expression in filamentous fungi.

8. A plasmid vector according to claims 1 to 7, wherein the nucleic acid sequence d) is functionally linked to a transcription terminator facilitating recombinant expression in filamentous fungi.

9. A selection marker comprising a nucleic acid sequence encoding a polyketide synthetase fragment, wherein said nucleic acid sequence comprises
i. a nucleic acid sequence shown in SEQ ID NO:1 or SEQ ID NO:2, SEQ ID NO:3, SEQ ID NO:4 or SEQ ID NO:5; or
ii. a nucleic acid sequence which, owing to the degeneracy of the genetic code, can be deduced from the amino acid sequence shown in SEQ ID NO:6 by back translation; or
iii. a functional equivalent of the nucleic acid sequence set forth in i) which is encoded by an amino acid sequence that has at least an identity of 50% with the SEQ ID NO: or from a functional equivalent of an amino acid sequence shown in SEQ ID NO:6 that has at least an identity of 41% with the SEQ ID NO:6 or from a functional equivalent of an amino acid sequence shown in SEQ ID NO:8 that has at least an identity of 49% with the SEQ ID NO:8 or from a functional equivalent of an amino acid sequence shown in SEQ ID NO:10 that has at least an identity of 6; or
iv. parts of the nucleic acid sequence as defined in i., ii. or iii. consisting of at least 300 bp; or
v. parts of the nucleic acid sequence as defined in i., ii. or iii. consisting of at least 300 bp comprising
a) a nucleic acid sequence shown in SEQ ID NO:7; or
b) a nucleic acid sequence which, owing to the degeneracy of the genetic code, can be deduced from the amino acid sequence shown in SEQ ID NO:8 by back translation; or
c) a functional equivalent of a nucleic acid sequence set forth in a), which is encoded by amino acid sequence that has at least an identity of 85% with the SEQ ID NO:8.

10. Use of a nucleic acid sequence comprising
a) a nucleic acid sequence encoding a polyketide synthetase; or
b) parts of the nucleic acid sequence as defined in i. consisting of at least 300 bp.
as marker for targeted transformation in filamentous fungi.

11. Use of a nucleic acid sequence according to claim 10 said nucleic acid sequence comprising
i. a nucleic acid sequence according to claim 9; or
ii. a nucleic acid sequence shown in SEQ ID NO:9 or SEQ ID NO: 11; or
iii. a nucleic acid sequence which, owing to the degeneracy of the genetic code, can be deduced from the amino acid sequence shown in SEQ ID NO:10, SEQ ID NO:12 or SEQ ID NO:13 by back translation; or
iv. a functional equivalent of the nucleic acid sequence set forth in i), which is encoded by an amino acid sequence that has at least an identity of 40% with the SEQ ID NO:6 or from a functional equivalent of an amino acid sequence shown in SEQ ID NO:6 that has at least an identity of 38% with the SEQ ID NO:6 or from a functional equivalent of an amino acid sequence shown in SEQ ID NO:8 that has at least an identity of 39% with the SEQ ID NO:8 or from a functional equivalent of an amino acid sequence shown in SEQ ID NO:10 that has at least an identity of; or
v. parts of the nucleic acid sequence as defined in ii., iii. or iv. consisting of at least 300 bp; or
vi. parts of the nucleic acid sequence as definied in ii., iii or iv. consisting of at least 300 bp comprising a nucleic acid sequence, which is encoded by an amino acid sequence that has at least an identity of 68% with the SEQ ID NO:8.

12. A plasmid vector for targeted transformation of filamentous fungi additionally comprising a selection marker comprising a nucleic acid sequence encoding a polyketide synthetase fragment, said nucleic acid sequence comprising
i. a nucleic acid sequence according to claim 9; or
ii. a functional equivalent of the nucleic acid sequence set forth in i) which is encoded by an amino acid sequence that has at least an identity of 40% with the SEQ ID NO:6.
iii. a nucleic acid sequence shown in SEQ ID NO:9 or SEQ ID NO: 11;
iv. a nucleic acid sequence which, owing to the degeneracy of the genetic code, can be deduced from the amino acid sequence shown in SEQ ID NO:10, SEQ ID NO:12 or SEQ ID NO:13 by back translation; or
v. parts of the nucleic acid sequence as defined in ii., iii. or iv. consisting of at least 300 bp; or
vi. parts of the nucleic acid sequence as defined in i., ii. or iii. or iv. consisting of at least 300 bp, which are encoded by an amino acid sequence that has at least an identity of 68% with SEQ ID NO:8.

13. A plasmid vector for targeted transformation of filamentous fungi as claimed in claims 1 to 8, additionally comprising a selection marker comprising a nucleic acid sequence encoding a polyketide synthetase fragment, said nucleic acid sequence comprising
i. a nucleic acid sequence according to claim 9; or
ii. a functional equivalent of the nucleic acid sequence set forth in i), which is encoded by an amino acid sequence that has at least an identity of 40% with the SEQ ID NO:6; or
iii. a nucleic acid sequence shown in SEQ ID NO:9 or SEQ ID NO: 11;
iv. a nucleic acid sequence which, owing to the degeneracy of the genetic code, can be deduced from the amino acid sequence shown in SEQ ID NO:10, SEQ ID NO:12 or SEQ ID NO:13 by back translation; or
v. parts of the nucleic acid sequence as defined in ii., iii. or iv. consisting of at least 300 bp; or
vi. parts of the nucleic acid sequence as defined in i., ii. or iii. or iv. consisting of at least 300 bp comprising a nucleic acid sequence, which is encoded by a functional equivalent of an amino acid sequence that has at least an identity of 68% with the SEQ ID NO:8.

14. An expression cassette comprising
a) a promotor sequence in functional linkage with a nucleic acid sequence according to claim 9 in antisense orientation; and optionally
b) further genetic control sequences functionally linked to a nucleic acid sequence according to a).

15. A plasmid vector for targeted transformation of filamentous fungi additionally comprising an expression cassette according to claim 14.

16. A plasmid vector for targeted transformation of filamentous fungi as claimed in claims 1 to 8, additionally comprising an expression cassette according to claim 14.

17. A method for transforming filamentous fungi, comprising the following steps
a) transferring a plasmid vector according to claim 12, 13, 15 or 16 into a filamentous fungi;
b) selecting successfully transformed filamentous fungi by the absence of color.

18. An expression cassette comprising
a) a promotor sequence in functional linkage with a nucleic acid sequence comprising
i. a nucleic acid sequence shown in SEQ ID NO:3, 4 or 5; or
ii. a nucleic acid sequence which, owing to the degeneracy of the genetic code, can be deduced from the amino acid sequence shown in SEQ ID NO:6 by back translation; or
iii. a functional equivalent of the nucleic acid sequence set forth in i) which is encoded by an amino acid sequence shown in SEQ ID NO:6 that has at least an identity of 40% with the SEQ ID NO:6; or
iv. a nucleic acid sequence shown in SEQ ID NO:9 or SEQ ID NO: 11;
v. a nucleic acid sequence which, owing to the degeneracy of the genetic code, can be deduced from the amino acid sequence shown in SEQ ID NO:10, SEQ ID NO:12 or SEQ ID NO:13 by back translation;
and optionally
b) further genetic control sequences functionally linked to a nucleic acid sequence according to a).

19. A method for transformation of filamentous fungi, comprising the following steps
a) providing a filamentous fungi, in which the polyketide synthetase gene is modified in such away that the polyketide synthetase cannot be functionally expressed;
b) transforming the filamentous fungi of step a) with an expression cassette according to claim 18 or a vector comprising the aforementioned expression cassette;
c) selecting successfully transformed filamentous fungi by the presence of color.

20. A method as claimed in claim 17 or 19, wherein the plasmid vector comprises at least an additional selection marker.

21. A method as claimed in claims 17, 19 or 20, wherein the selection is confirmed by PCR.

22. A method as claimed in claims 17, 19, 20 or 21 wherein the filamentous fungi are succesuflly transformed and identified in a high-throughput screening.
